(19) United States
(12) Patent Application Publication
(10) Pub. No.: US 2024/0423799 A1
(43) Pub. Date: Dec. 26, 2024

(54) CARDIAC VALVE ENHANCEMENT APPLIANCES

(71) Applicant: Allan C. Entis, Tel Aviv (IL)

(72) Inventor: Allan C. Entis, Tel Aviv (IL)

(21) Appl. No.: 18/699,280

(22) PCT Filed: Oct. 6, 2022
(86) PCT No.: PCT/IL2022/051062

§ 371 Date: Apr. 7, 2024

Related U.S. Application Data
(60) Provisional application No. 63/304,636, filed on Jan. 30, 2022.
(60) Provisional application No. 63/252,707, filed on Oct. 6, 2021.

(51) Int. Cl. A61F 2/24

(52) U.S. Cl. CPC A61F 2/246; A61F 2210/0014; A61F 2220/0008; A61F 2230/0013; A61F 2230/0039

(57) ABSTRACT

Apparatus for affecting operation of a cardiac valve of a heart the valve having cardiac valve leaflets, the enhancer comprising: an annular cardiac brace for mounting to an annulus of a cardiac valve and a scaffolding connected to the brace and comprising at least one extendable scaffolding strut; wherein the apparatus is deformable from a delivery configuration to a deployed configuration and in the delivery configuration the top wings are oriented substantially back to back along an axis, and the bottom wings are oriented substantially back to back along the same axis and the at least one extendable scaffolding strut lies along the top or bottom wings, and in the deployed configuration the first top and bottom gripping wings face each other to grip the annulus between them and the second top and bottom gripping wings face each other to grip the annulus between them and the at least one extendable scaffolding arm extends away from the wings into a chamber of the heart on a retrograde side of the valve or an antegrade side of the valve.

### TECHNICAL FIELD

Embodiments of the disclosure relate to devices and instruments for implementing cardiac valve corrective surgery.

### BACKGROUND

The human heart, and generally all mammalian hearts, comprises two blood pumps that operate in synchrony to oxygenate and deliver oxygenated blood to the body. A first pump receives deoxygenated blood after it has coursed through blood vessels in the circulatory system to deliver oxygen and nutrients to the various parts the body and pumps the deoxygenated blood through the lungs to be oxygenated. The second pump receives the oxygenated blood from the lungs and pumps it to flow through the blood vessels of the circulatory system and deliver oxygen and nutrients to the body parts. The two pumps are located adjacent each other in the heart and each pump comprises two chambers, an atrium that receives blood and a ventricle that pumps blood.

The first pump, which receives deoxygenated blood to be pumped to the lungs, is located on the right side of the heart and its atrium and ventricle are accordingly referred to as the right atrium and right ventricle. The second pump, which receives oxygenated blood to be pumped to the body, is located on the left side of the heart and its atrium and ventricle are referred to as the left atrium and left ventricle of the heart. The right and left atria are separated by a wall in the heart referred to as the interatrial septum and the right and left ventricles are separated by a wall in the heart referred to as the interventricular septum.

Deoxygenated blood enters the right atrium via blood vessels referred to as the superior vena cava and inferior vena cava. During a part of the heart cycle referred to as diastole the right ventricle is relaxed and the deoxygenated blood in the right atrium flows from the right atrium into the right ventricle via a valve, referred to as a tricuspid valve, which connects the right atrium to the right ventricle. The right ventricle contracts during a part of the heart cycle referred to as systole, to pump the deoxygenated blood that it receives from the right atrium out of the ventricle and into the pulmonary artery via a valve referred to as the pulmonary valve, which interfaces the pulmonary artery with the right ventricle. The pulmonary artery delivers the deoxygenated blood to the lungs for oxygenation. The tricuspid and pulmonary valves control direction of blood flow in the right side of the heart. The tricuspid valve opens to let deoxygenated blood flow from the right atrium into the right ventricle and closes to prevent deoxygenated blood from regurgitating into the right atrium when the right ventricle contracts. The pulmonary valve opens to let blood enter the pulmonary artery when the right ventricle contracts and closes to prevent blood regurgitating into the right ventricle when the right ventricle relaxes to receive blood from the right atrium.

The left atrium receives oxygenated blood from the lungs via pulmonary veins. Oxygenated blood flows from the left atrium into the left ventricle during diastole via a bileaflet valve referred to as the mitral valve, which opens during diastole to allow blood flow from the left atrium to the left ventricle. The left ventricle contracts during systole to pump the oxygenated blood that it receives from the left atrium out of the heart through the aortic valve and into the aorta, for delivery to the body. The mitral valve operates to prevent regurgitation of oxygenated blood from the left ventricle to the left atrium when the left ventricle contracts to pump oxygenated blood into the aorta. The aortic valve closes to prevent blood from regurgitating into the left ventricle when the left ventricle relaxes to receive blood from the left atrium.

Each valve comprises a set of matching “flaps”, also referred to as “leaflets” or “cusps”. that are mounted to and extend from a supporting structure of fibrous tissue. The supporting structure has a shape reminiscent of an annulus and is often conventionally referred to as the annulus of the valve. The leaflets are configured to align and overlap each other, or coapt, along free edges of the leaflets to close the valve. The valve opens when the leaflets are pushed away from each other and their free edges part. The aortic, pulmonary, and tricuspid valves comprise three leaflets. The mitral valve comprises two leaflets.

The leaflets in a valve open and close in response to a gradient in blood pressure across the valve generated by a difference between blood pressure on opposite sides of the valve. When the gradient is negative in a “downstream flow” or antegrade direction, in which direction the valve is intended to enable blood flow, the leaflets are pushed apart in the downstream, antegrade direction by the pressure gradient, and the valve opens. When the gradient is positive in the upstream direction, the leaflets are pushed together in the upstream or retrograde direction so that their respective edges meet to align and coapt, and the valve closes.

For example, the leaflets in the mitral valve are pushed apart during diastole to open the mitral valve and allow blood flow from the left atrium into the left ventricle when pressure in the left atrium is greater than pressure in the left ventricle. The leaflets in the mitral valve are pushed together so that their edges coapt to close the valve during systole when pressure in the left ventricle is greater than pressure in the left atrium to prevent regurgitation of blood into the left atrium.

Each valve is configured to prevent misalignment or prolapse of its leaflets as a result of positive pressure gradients pushing the leaflets upstream past a region in which the leaflets properly align and coapt to close the valve. A construction of fibrous tissue in the leaflets of the pulmonary and aortic valves operates to prevent prolapse of the leaflets in the pulmonary and aortic valves. A configuration of cord-like tendons, referred to as chordae tendineae, connected to muscular protrusions, referred to as papillary muscles, that project from the left ventricle wall, tie the leaflets of the mitral valve to the walls of the left ventricle. The chordae tendineae provide dynamic anchoring of the mitral valve leaflets to the left ventricle wall that operate to limit upstream motion of the leaflets and prevent their prolapse into the left atrium during systole. Similarly, a configuration of chordae tendineae and papillary muscles cooperate to prevent prolapse of the tricuspid valve leaflets into the right atrium

Efficient cardiac valve function can be complex and a cardiac valve may become compromised by disease or injury to an extent that warrants surgical intervention to effect its repair or replacement. For example, normal mitral valve opening and closing and prevention of regurgitation of blood from the left ventricle into the left atrium is dependent on coordinated temporal cooperation of the mitral leaflets, the mitral annulus, the chordae, papillary muscles, left ventricle, and left atrium. Malfunction of any of these components of a person's heart may lead to mitral valve dysfunction and regurgitation that warrants surgical intervention to provide the person with an acceptable state of health and quality of life.

### SUMMARY

An aspect of an embodiment of the disclosure relates to providing a structure and procedures for emplacing and securing the structure to an annulus of a cardiac valve that may operate to improve functioning of the valve, and/or provide structural support for apparatus that operates to improve functioning of the valve. According to an aspect of an embodiment of the disclosure, the structure, comprises a cardiac “annular brace” or a “brace”, that is configured to grip and anchor the structure to an annulus of a cardiac valve in a region of a commissure of the valve. In an embodiment the structure comprises a scaffolding comprising at least one strut that extends from the brace to improve or cooperate with additional devices attached to the scaffolding to improve functioning of the valve. Hereinafter the structure may be referred to as a cardiac valve enhancer, or simply enhancer.

In an embodiment of the disclosure, the enhancer annular brace comprises first and second bottom gripping wings and first and second top gripping wings for gripping and anchoring to the annulus of the cardiac valve in a region of the commissure. The top gripping wings are connected to the bottom gripping wings by a support bridge. The scaffolding is connected to at least one wing and/or the support bridge. The enhancer has a delivery configuration and a deployed configuration and is formed from a suitable deformable biocompatible material so that the enhancer is deformable from the delivery configuration to the deployed configuration. Optionally, the deformable biocompatible material is a shape memory alloy such as nitinol. In the delivery configuration the shape memory alloy brace may be in a martensite state and in the deployed configuration the material may be in an austenite state.

In the delivery configuration the first and second bottom gripping wings are folded to lie substantially back to back along a same axis and the first and second top gripping wings are folded to lie substantially back to back along the same axis along which the bottom gripping wings lie. In the delivery configuration the scaffolding also lies along the axis. In the deployed configuration, the first top and first bottom gripping wings oppose each other to grip a first region of the annulus between them, and the second top and second bottom gripping wings oppose each other to grip a second region of the annulus between them. The gripping wings are shaped to conform to the curvature of the annular regions that they grip. The first and second regions of the annulus lie on opposite sides of the commissure. In the deployed configuration, the scaffolding extends away from the brace optionally by unfolding from the brace or as a result of the brace unfolding to assume the deployed configuration.

A deployment catheter houses the cardiac enhancer in the delivery configuration for delivery to and for positioning the enhancer for deployment at the cardiac valve. Following delivery and positioning at the commissure, the deployment catheter is controlled to release the enhancer so that the enhancer may deform to the deployed state in which the brace grips and anchors to the annulus and the scaffolding extends away from the brace.

In an embodiment of the disclosure, the top and bottom first gripping wings are integral portions of a same first “gripping strip” of an elastically deformable biocompatible material and the top and bottom second gripping wings are integral portions of a second gripping strip of an elastically deformable biocompatible material. Optionally, the scaffolding is also an integral portion of the first or second gripping strip. A bridge portion of the first gripping strip located between the first gripping strip's top and bottom wings is connected to a bridge portion of the second gripping strip located between the second gripping strip's top and bottom gripping wings to form the bridge connecting both top wings to both bottom wings.

In the delivery configuration of the cardiac enhancer the strips may be flat and lie substantially back to back. In the deployed configuration the strips are bent so that the top and bottom wings of the first strip face each other to grip the first region of the annulus and the top and bottom wings of the second strip face each other to grip the second region of the annulus. The deployment catheter that houses the enhancer for delivery to the annulus optionally constrains the enhancer to the enhancer's delivery configuration. Upon release from the deployment catheter, the enhancer, optionally, self-deforms to the brace's deployed configuration.

In an embodiment of the disclosure the first and second gripping strips are separate strips and their respective bridge portions are connected using any of various suitable joining process, such as bonding, gluing, welding, or brazing. Optionally, the first and second gripping strips the bridge and scaffolding are integral parts of a same piece, hereinafter also referred to as a “die-shape”, of material shaped by a process such as by way of example, stamping or laser cutting from a sheet of an elastically deformable biocompatible material. Suitably bending the die-shape deforms the die-shape to the delivery configuration of the enhancer.

In an embodiment of the disclosure, the annular brace comprises a wireform that is bent to provide the first and second top and bottom wings. Optionally, the wireform is cut or stamped from a sheet of an elastically deformable biocompatible material.

In an embodiment a cardiac enhancer comprises a scaffolding, referred to as a cardiac valve leaflet scaffolding (CAVALS), which in the enhancer delivery configuration comprises at least one scaffolding strut that is folded to the enhancer brace to enable the enhancer to be delivered by a delivery catheter for deployment to a cardiac valve at which the CAVALS is to be deployed. In the deployed configuration the CAVALS unfolds away from the annular brace to limit motion of a leaflet of the valve and thereby moderate or eliminate regurgitation. In an embodiment the at least one scaffolding strut is configured to support a component, such as a tissue or fabric, that operates to prevent or moderate leaking of blood through the leaflet or a seam between leaflets of the valve at which the leaflets do not coapt properly.

In an embodiment a cardiac enhancer scaffolding comprises a cardiac valve leaflet depressor, optionally referred to by the acronym CVLD an extendable depressor. In the delivery configuration of the enhancer the depressor is folded along a top gripping wing of the brace and in the deployed configuration extends away from the top gripping wing to press on a desired region of a leaflet of the valve to moderate motion of the leaflet and improve valve functioning. Optionally, the leaflet depressor has a paddle shape.

An aspect of an embodiment of the disclosure relates to providing an enhancer optionally referred to as a cardiac valve neochord “emplacer”, or simply emplacer that is usable to implant a neochord into the heart to tether a leaflet of a cardiac valve, optionally a mitral valve. In an embodiment the neochord emplacer comprises a brace having an extension, also referred to as an anchor tail, to which a prosthetic neochord is attached. In the delivery configuration the anchor tail extends along the bottom gripping wings of the annular brace and the neochord extends along a delivery catheter used to deliver the emplacer from a point at which it is attached to the anchor tail to a proximal end of the delivery catheter. When deployed for tethering a leaflet of a mitral valve, the anchor tail extends into the left ventricle. With the delivery catheter at least partially retracted and the neochord extending between the coaptation edges of the mitral valve leaflets, a leaflet catcher, may then be slid along the neochord to a location along a coaptation edge of one of the leaflets and released to clamp to the neochord and the leaflet to anchor the neochord along the coaptation edge of the leaflet.

An aspect of an embodiment of the disclosure related to providing a cardiac enhancer, hereinafter also referred to as a cardiac modulator (CARMO), that may be deployed in the heart to modulate structure and motion of the heart and improve functioning of a cardiac valve. A CARMO in accordance with an embodiment, optionally comprises an annular brace that supports a scaffolding having at least one modulator strut that lies folded along a top or bottom griping wing of the brace in a delivery configuration of the CARMO. In a deployed configuration of the CARMO, the brace is mounted to an annulus of a cardiac valve, optionally the mitral valve, and the at least one modulator strut extends away from the valve to press and exert force against a wall of a chamber of the heart. When deployed to the mitral valve, the at least one brace extends to press against the wall of the left ventricle and/or the left atrium to exert force that may operate to modify shape and/or dynamic motion of the annulus and/or valve leaflets.

An aspect of an embodiment of the disclosure related to providing a cardiac enhancer, also referred to as a “shim seater” that may be deployed in the heart to position, or seat, a layer of material, hereinafter referred to as a “leaflet shim”, having advantageous thickness between coaptating edges of a mitral valve to facilitate sealing of the edges to prevent regurgitation.

In the discussion, unless otherwise stated, adjectives such as “substantially” and “about” modifying a condition or relationship characteristic of a feature or features of an embodiment of the disclosure, are understood to mean that the condition or characteristic is defined to within tolerances that are acceptable for operation of the embodiment for an application for which it is intended. Wherever a general term in the disclosure is illustrated by reference to an example instance or a list of example instances, the instance or instances referred to, are by way of non-limiting example instances of the general term, and the general term is not intended to be limited to the specific example instance or instances referred to. The phrase “in an embodiment”, whether or not associated with a permissive, such as “may”, “optionally”, or “by way of example”, is used to introduce for consideration an example, but not necessarily a required configuration of possible embodiments of the disclosure. Unless otherwise indicated, the word “or” in the description and claims is considered to be the inclusive “or” rather than the exclusive or, and indicates at least one of, or any combination of more than one of items it conjoins.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF FIGURES

Non-limiting examples of embodiments of the disclosure are described below with reference to figures attached hereto that are listed following this paragraph. Identical features that appear in more than one figure are generally labeled with a same label in all the figures in which they appear. A label labeling an icon representing a given feature of an embodiment of the disclosure in a figure may be used to reference the given feature. Dimensions of features shown in the figures are chosen for convenience and clarity of presentation and are not necessarily shown to scale.

FIG. 1 schematically shows a cross section of a human heart that displays the heart chambers and cardiac valves;

FIG. 2A schematically shows a cutaway perspective view of a human heart that provides a perception of the three dimensional structure of the mitral and tricuspid valves of the heart;

FIG. 2B schematically shows the cutaway perspective view of a human heart similar to that shown in FIG. 2A with the anterior leaflet of the mitral valve cutaway for convenience of presentation;

FIG. 3 schematically shows an annular brace mounted to the annulus of the mitral valve shown in FIG. 2B, in accordance with prior art;

FIGS. 4A-4C schematically illustrate construction of the annular brace shown deployed in FIG. 3, in accordance with prior art;

FIGS. 4D and 4E schematically show variations of annular braces similar to the annular brace shown in FIGS. 3 and 4A-4C, in accordance with prior art;

FIGS. 4F-4H schematically show stages in deployment of the brace shown in FIG. 3 in accordance with prior art;

FIG. 4I schematically shows an annular brace having stabilizer teeth that function to facilitate stabilization of deployment of an annular brace during deployment, in accordance with prior art;

FIGS. 5A-5D schematically illustrate a transseptal deployment of the annular brace shown in FIGS. 3 and 4A-4G, in accordance with prior art;

FIG. 6 schematically shows a view from the atrial side of a mitral valve having an annular brace mounted at each commissure of the valve and leaflet restraining struts mounted to the braces, in accordance with prior art;

FIGS. 7A-7D schematically show a wireframe annular brace, in accordance with prior art;

FIGS. 8A-FIG. 11G schematically show various configurations of a cardiac valve enhancer comprising a scaffolding, in accordance with an embodiment of the disclosure;

FIGS. 12A-12D schematically show a cardiac valve enhancer comprising a leaflet depressor (CVLD), in accordance with an embodiment of the disclosure;

FIGS. 13A-13E schematically show a cardiac valve enhancer also referred to as a cardiac valve neochord emplacer comprising a neochord and leaflet catcher, in accordance with an embodiment of the disclosure;

FIGS. 13F schematically show a leaflet catcher for use in clamping a neochord to a cardiac valve leaflet, in accordance with an embodiment of the disclosure;

FIGS. 13G-13I schematically show a leaflet catcher positioner for use in determining where along a neochord to clamp the neochord to a cardiac valve leaflet, in accordance with an embodiment of the disclosure;

FIGS. 14A-14E schematically show a cardiac valve enhancer also referred to as a cardiac modulator (CARMO) that may be deployed in the heart to modulate structure and motion of the heart and improve functioning of a cardiac valve, in accordance with an embodiment of the disclosure; and

FIGS. 15A-15C schematically show a cardiac valve enhancer also referred to as a shim seater that may be deployed in the heart to seat a layer of material, a “leaflet shim”, between coaptating edges of a mitral valve to facilitate sealing of the edges to prevent regurgitation, in accordance with an embodiment of the disclosure.

### DETAILED DESCRIPTION

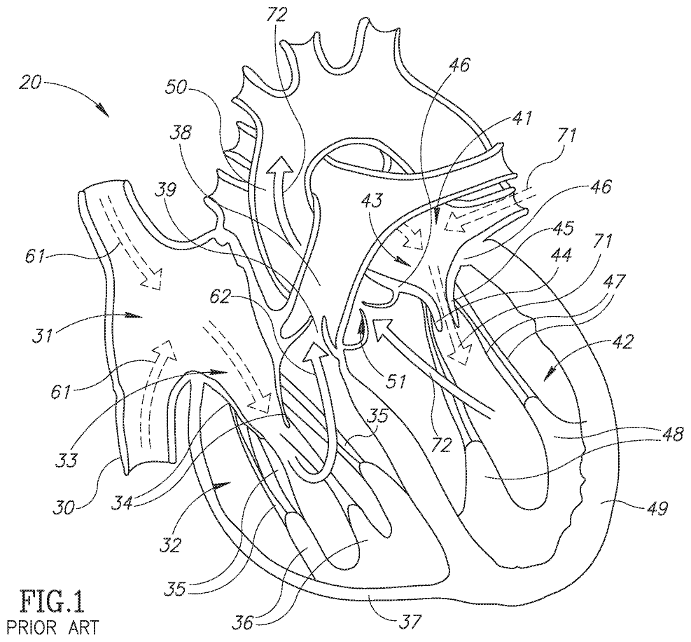

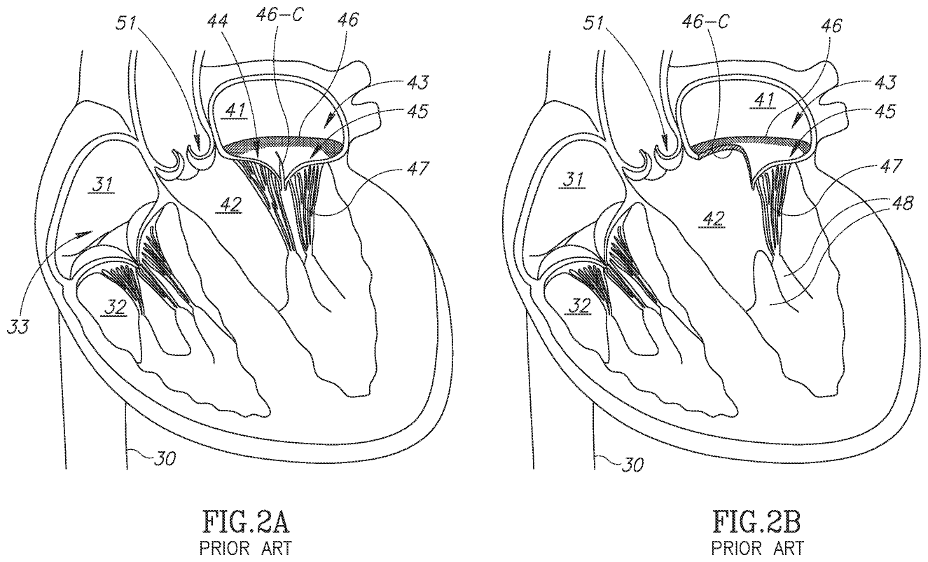

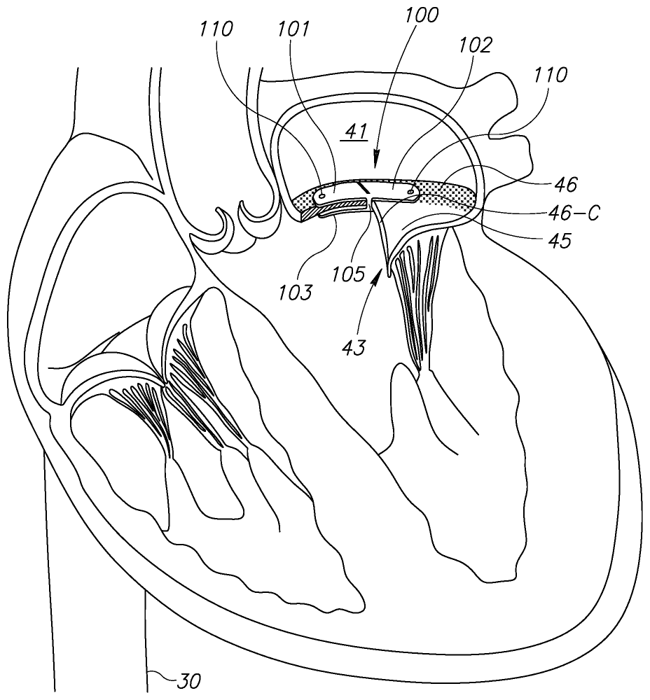

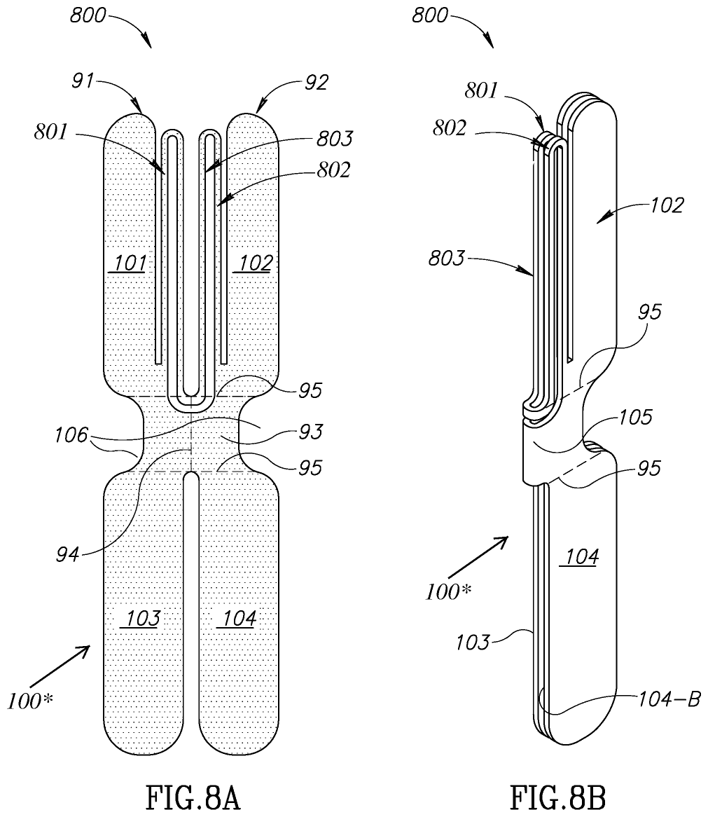

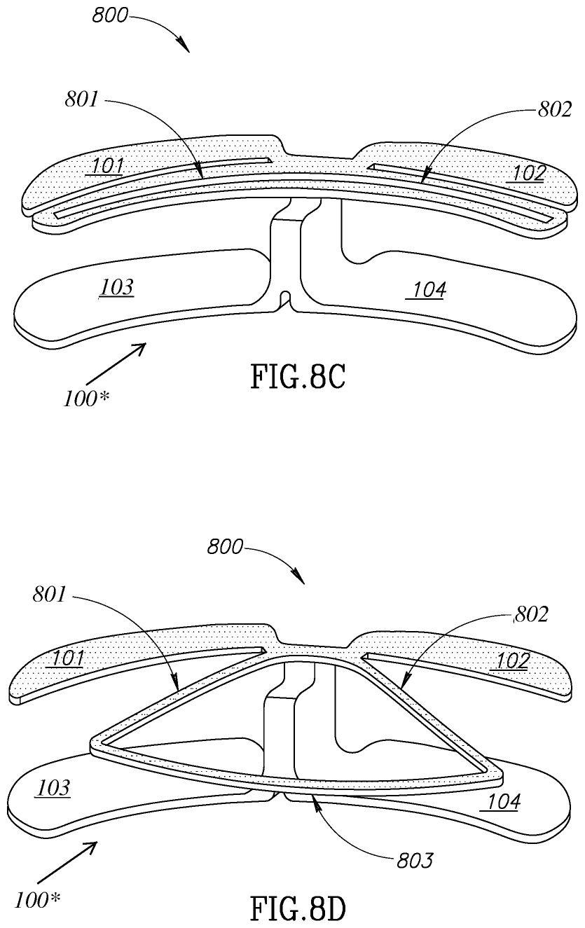

In the following detailed description structure and operation of cardiac valves of a human heart are discussed with reference to FIGS. 1-2B. Structure, manufacture, and deployment of a cardiac annular brace in accordance with prior art are discussed with respect to FIGS. 3-7D. Cardiac enhancers in accordance with embodiments of the disclosure are discussed with reference to FIGS. 8A-17C. FIGS. 8A-17C schematically illustrate structure, manufacture, and deployment of cardiac enhancers comprising an annular brace, such as a brace discussed with reference to FIGS. 3-7D, in accordance with embodiments of the disclosure.

FIG. 1 shows a schematic, stylized cross section of a human heart 20 having a right atrium 31 and a right ventricle 32 that communicate via a tricuspid valve 33 and a left atrium 41 and left ventricle 42 that communicate via a mitral valve 43. Tricuspid valve 33 has three leaflets 34, only two of which are shown in FIG. 1, that are tied by chordae tendineae 35 and papillary muscles 36 to the wall 37 of the right ventricle. Right ventricle 32 communicates with the pulmonary artery 38 via the pulmonary valve 39. Mitral valve 43 has two leaflets, anterior and posterior leaflets 44 (anterior leaflet 44 is in continuity with the wall of the aorta) and 45 respectively that are supported and extend from the mitral annulus 46. Mitral valve leaflets 44 and 45 are respectively tied by chordae tendineae 47 and papillary muscles 48 to the ventricle wall 49. The left ventricle communicates with the aorta 50 via the aortic valve 51.

Deoxygenated blood returning from parts of the body enters right atrium 31 and passes through tricuspid valve 33 to enter right ventricle 32 during diastole when leaflets 34 of the tricuspid valve are separated (as schematically shown in FIG. 1) to open the tricuspid valve and the right ventricle is relaxed. Flow of deoxygenated blood into the right atrium via the inferior vena cava 30 and through tricuspid valve 33 into the right ventricle is schematically indicated by dashed line block arrows 61. During systole right ventricle 32 contracts to pump the deoxygenated blood through pulmonary valve 39 and into pulmonary artery 38 for delivery to the lungs. During systole leaflets 34 of tricuspid valve 33 coapt and the tricuspid valve 33 closes to prevent deoxygenated blood pumped by the right ventricle from regurgitating into the right atrium. Flow of deoxygenated blood pumped by right ventricle 32 into pulmonary artery 38 is schematically indicated by solid line block arrow 62.

Oxygenated blood from the lungs enters left atrium 41 and passes through mitral valve 43 to enter left ventricle 42 during diastole when leaflets 44 and 45 are separated (as shown in FIG. 1) to open the mitral valve and the left ventricle is relaxed. Flow of oxygenated blood into the left atrium and through mitral valve 43 into the left ventricle is schematically indicated by dashed block arrows 71. During systole left ventricle 42 contracts to pump the oxygenated blood through the aortic valve 51 and into the aorta 50 for delivery to the body. During systole leaflets 44 and 45 coapt to close mitral valve 43 and prevent oxygenated blood pumped by the left ventricle from regurgitating into the left atrium.

Valves 33, 39, 43, and 51 operate to direct flow of blood in the heart and out from the heart and their proper and efficient function are required to maintain a person's health and quality of life. Various different disease processes may result in damage to a heart valve and compromise valve functioning. For example, functioning of the mitral valve may be compromised by various degrees of stenosis, calcification, distortion of the mitral valve annulus, torn chordae tendineae, and faulty left ventricle functioning. Valve dysfunction and concomitant regurgitation may become so severe as to warrant surgical intervention to provide a person with an acceptable state of health and quality of life.

FIG. 2A schematically shows a cutaway perspective view of a human heart 20 that provides a perception of the three dimensional structure of mitral valve 43 and tricuspid valve 33. In the figure, a portion of annulus 46 of mitral valve 43 that supports mitral valve anterior and posterior leaflets 44 and 45 is shown shaded, and a region of a commissure 46-C at which the leaflets come together is indicated. For convenience of presentation and the discussion that follows, FIG. 2B schematically shows heart 20 in which anterior leaflet 44 shown in FIG. 2A is cutaway substantially to annulus 46, and chordae tendineae 47 that connect the anterior leaflet to papillary muscles 48 are removed.

FIG. 3 schematically shows an annular brace 100 mounted to annulus 46 in the region of commissure 46-C in accordance with an embodiment of the disclosure. Annular brace 100 comprises first and second top gripping wings 101 and 102 and first and second bottom gripping wings 103 and 104. In the perspective of FIG. 3 bottom gripping wing 104 is hidden by posterior leaflet 45 and is not shown. Top gripping wings 101 and 102 are joined to bottom gripping wings 103 and 104 by a bridge 105. Optionally, top gripping wings 101 and 102 are formed having mounting holes 110 for mounting apparatus to annular brace 100 that a medical practitioner may determine to be advantageous for ameliorating a mitral valve dysfunction.

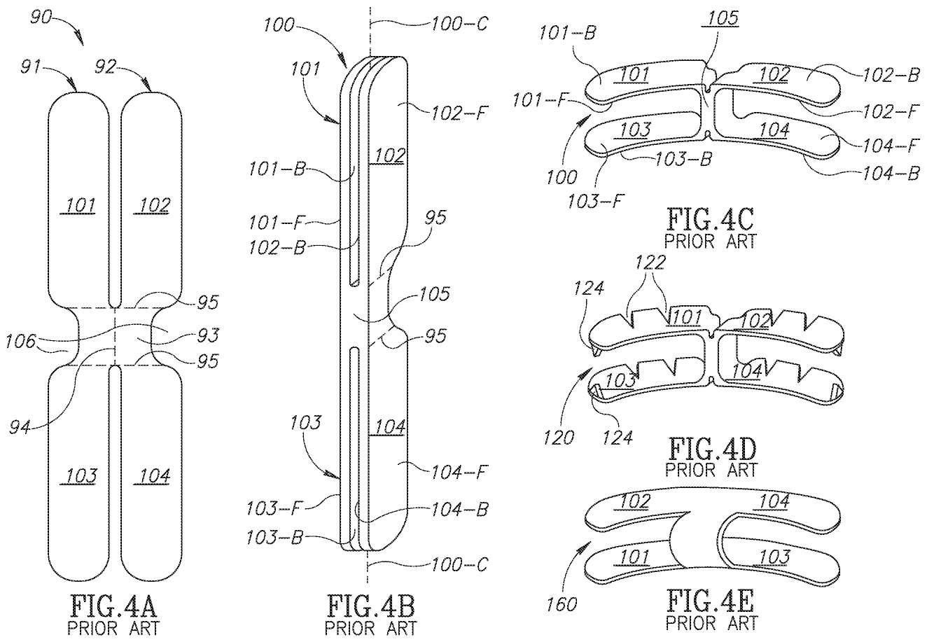

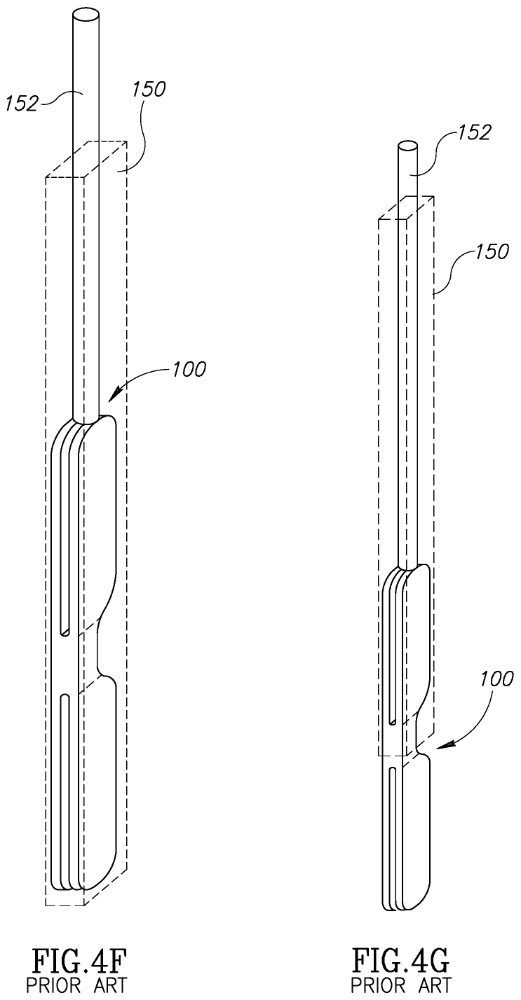

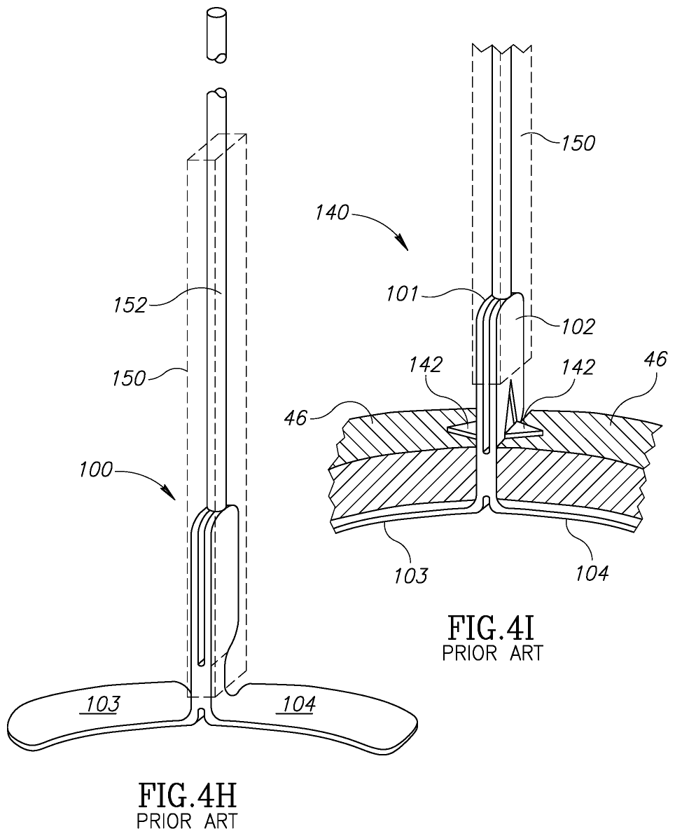

FIGS. 4A-4H schematically illustrate construction and stages in a deployment of annular brace 100 shown in FIG. 3, in accordance with an embodiment of the disclosure. Brace 100 is optionally formed from a planar die-shape 90, schematically shown in FIG. 4A. The die-shape optionally comprises a gripping strip 91 having top gripping wings 101 and 102, and a gripping strip 92 having bottom gripping wings 103 and 104. A bridging region 93 connects gripping strips 91 and 92. Optionally, die-shape 90 is “scalloped” to produce recesses 106 in which annulus 46 seats when brace 100 is fully deployed as shown in FIG. 3. A dashed line 94 on die-shape 90 indicates a fold line along which bridging region 93 is folded to form bridge 105 (FIG. 3). Folding bridging region 93 along fold line 94 produces annular brace 100 in the brace's delivery configuration as schematically shown in FIG. 4B. Dashed lines 95 shown in FIGS. 4A and 4B indicate bend lines along which gripping wings 101-104 bend to deform brace 100 from the delivery configuration of the brace to the deployed configuration of the brace in which the brace grips annulus 46, as shown in FIG. 3. FIG. 4C shows annular brace 100 as it appears in the deployed configuration, without annulus 46.

It is noted, as shown in FIGS. 3 and 4C, that in the deployed state, gripping wings 101-104 not only bend along bend lines 95 but also optionally deform to conform to a curvature of annulus 46 at the location of commissure 46-C at which the brace is deployed. In an embodiment of the disclosure, to enable sufficient curvature of top and bottom gripping wings 101-104 to conform to curvature of a cardiac valve annulus, such as annulus 46 of mitral valve 43 or an annulus of a tricuspid valve, to which an annular brace, similar to annular brace 100, is mounted, the gripping wings may be slotted. Optionally, to facilitate anchoring brace 100 to the cardiac valve annulus, a gripping wing of the brace is formed having an anchor tooth that bites into the annulus when the gripping wing is deployed. In an embodiment of the disclosure, each wing of the annular brace is fitted, optionally at an end of the gripping wing, with at least one anchor tooth. By way of example FIG. 4D schematically shows an annular brace 120 similar to annular brace 100, in which gripping wings 101-104 are formed having slots 122 that facilitate curving of the gripping wings to conform to curvature of a cardiac valve annulus. Each wing 101-104 comprises an anchoring tooth 124 at an end of the wing.

Die-shape 90 (FIG. 4A) may be produced by way of example, by sintering, molding, or by cutting or stamping, from a sheet of a suitable elastically deformable material. Optionally, the material is a shape memory material which may be a shape memory alloy, such as nitinol, or a shape memory polymer. In an embodiment of the disclosure, the shape memory material is in a martensite state when brace 100 is in the delivery configuration and is in an austenite state when the brace deforms to the deployed configuration, which is a configuration the shape memory material is conditioned to remember. Any of various methods known in the art may be used to condition brace 100 to remember the deployed configuration. It is noted that a die-shape similar to die-shape 90 may be folded and conditioned to provide an annular brace 160 schematically shown in FIG. 4E that is similar to annular braces 100 and 120, but in which top gripping wings are gripping wings 102 and 104 and bottom gripping wings are gripping wings 101 and 103.

FIGS. 4F-4I schematically show stages in the delivery of brace 100 to mitral valve 43, in accordance with an embodiment of the disclosure. In FIG. 4F brace 100 is housed in a deployment catheter 150 schematically indicated in dashed lines. Optionally deployment catheter 150 has a rectangular or square cross section so that the catheter constrains brace 100 to the brace's delivery configuration as long as the brace is confined by the catheter. Optionally, deployment catheter 150 comprises a push rod 152 that is controllable to push brace 100 out from deployment catheter 150 and deploy the brace at mitral valve 43. FIG. 4G schematically shows annular brace 100 after push rod 152 has partially pushed the brace out of deployment catheter 150 to release bottom gripping wings 103 and 104 from the deployment catheter. Upon being pushed out from deployment catheter 150, and released from confinement by the deployment catheter, bottom gripping wings 103 and 104 bend from bridge 105 away from each other to their deployed orientation as schematically shown in FIG. 4H. Upon push rod 152 pushing annular brace 100 completely out of deployment catheter 150, top gripping wings 101 and 102 bend to their deployed state as shown in FIG. 4C and grip annulus 46 as shown in FIG. 3.

FIGS. 4F-4I show delivery catheter 150 fitting snugly to brace 100 and constraining the brace to a delivery configuration in which the brace appears to fit snugly in a rectangular volume. However, in an embodiment of the disclosure, delivery catheter 150 may have a sufficiently large cross section to allow a brace, similar to brace 100 to be delivered in a delivery configuration that is partially deformed from the delivery configuration shown in FIG. 4B and 4F to the deployed configuration shown in FIG. 4C.

By way of a numerical example, die shape 90 may have a thickness between about 0.5 mm to about 3 mm, wings 101-104 may lengths between about 5 mm to about 20 mm and widths between about 2 mm and about 5 mm. Delivery catheter may have an internal diameter up to about 7.5 mm.

In some embodiments of the disclosure an annular brace similar to annular brace 100 may have stabilizer teeth that deploy from top gripping wings as the annular brace is pushed out of deployment catheter 150 after bottom gripping wings 103 and 104 are deployed and a portion, but not all, of top gripping wings 101 and 102 are released from the deployment catheter. The stabilizer teeth aid in maintaining position of the annular brace during deployment of the brace. FIG. 4I schematically shows an annular brace 140 having stabilizer teeth 142 deployed on an upper surface of annulus 46 after bottom gripping wings 103 and 104 are deployed and top gripping wings 101 and 102 are partially extended from deployment catheter 150.

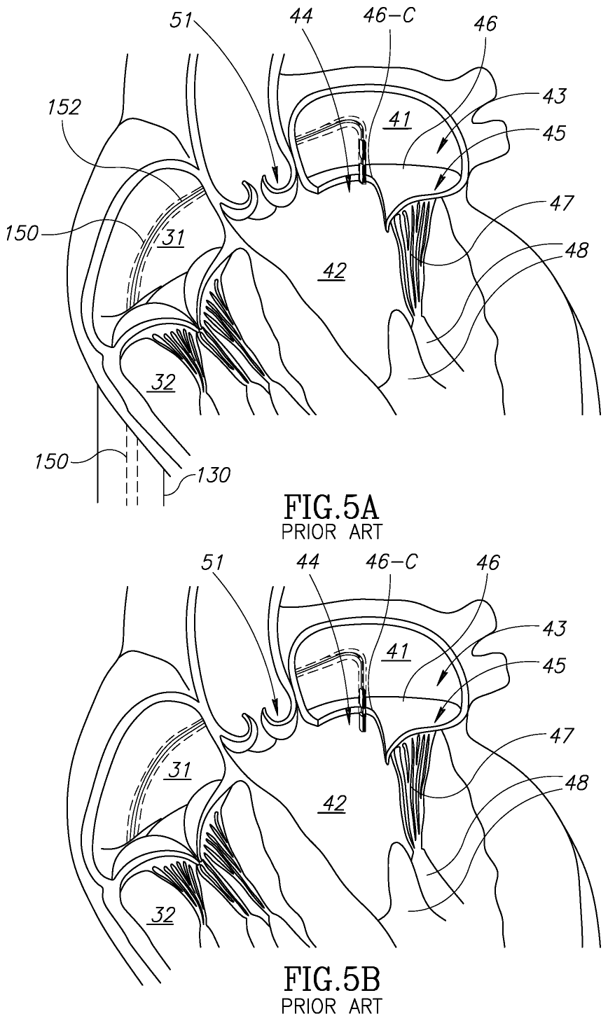

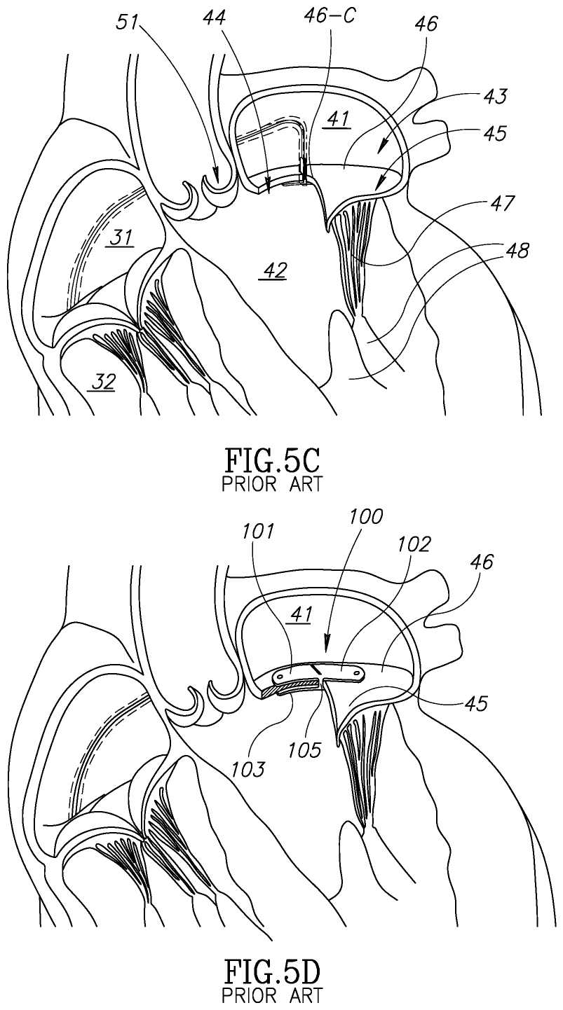

FIGS. 5A-5D schematically show a transseptal procedure for deploying annular brace 100 at mitral valve 43, in accordance with an embodiment of the disclosure. FIG. 5A schematically shows deployment catheter 150 after the deployment catheter has been threaded into right atrium 31 via the inferior vena cava 30 (FIGS. 1-2B) and been delivered through a puncture in the atrial septum (not shown) into the left atrium. In the left atrium, deployment catheter 150 has been controlled to position annular brace 100 housed in the catheter for mounting to annulus 46. In FIG. 5A annular brace 100 is housed in catheter 150 as shown in FIG. 4F. In FIG. 5B push rod 152 has been controlled to push annular brace 100 out from deployment catheter 150 so that bottom gripping wings 103 and 104 protrude between anterior and posterior leaflets 44 and 45 (FIGS. 1 and 2A) in a region of commissure 46-C to below the leaflets, and recess 106 cups the annulus. The position of bottom gripping wings 103 and 104 in FIG. 5B relative to deployment catheter 150 is similar to that shown in FIG. 4G. Having been released from deployment catheter 150, gripping wings 103 and 104 bend apart to their respective deployment locations under annulus 46 to the left and right respectively of commissure 46-C as schematically shown in FIG. 5C. In FIG. 5D push road 152 has pushed annular brace 100 completely out of deployment catheter 150, top gripping wings 101 and 102 have bent to their deployed configuration opposite gripping wings 103 and 104 respectively and deployment catheter 150 has been removed from left atrium 41. Top and bottom gripping wings 101 and 103 sandwich, grip, and anchor to a region of annulus 46 between them and top to the left of commissure 46-C and bottom gripping wings 102 and 104 sandwich, grip, and anchor to a region of annulus 46 between them to the right of commissure 46-C. Annular brace 100 is fully deployed and mounted to annulus 46 at commissure 46-C.

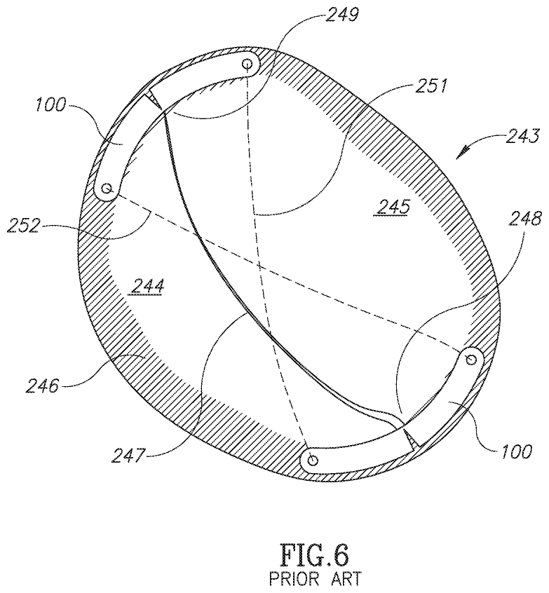

FIG. 6 schematically shows a view from the atrial side of a mitral valve 243 comprising leaflets 244 and 245 that are supported by an annulus 246 shown shaded and operate to coapt along a seam 247 that extends between commissures 248 and 249. Function of mitral valve 243 is assumed to be compromised by prolapse of leaflets 244 and 245 into the atrium. To alleviate prolapse, in accordance with an embodiment of the disclosure, an annular brace 100 is mounted to annulus 246 at each commissure 248 and 249 of the valve, and leaflet restraining struts 251 and 252 are mounted to braces 100 to restrain motion of the leaflets into the atrium.

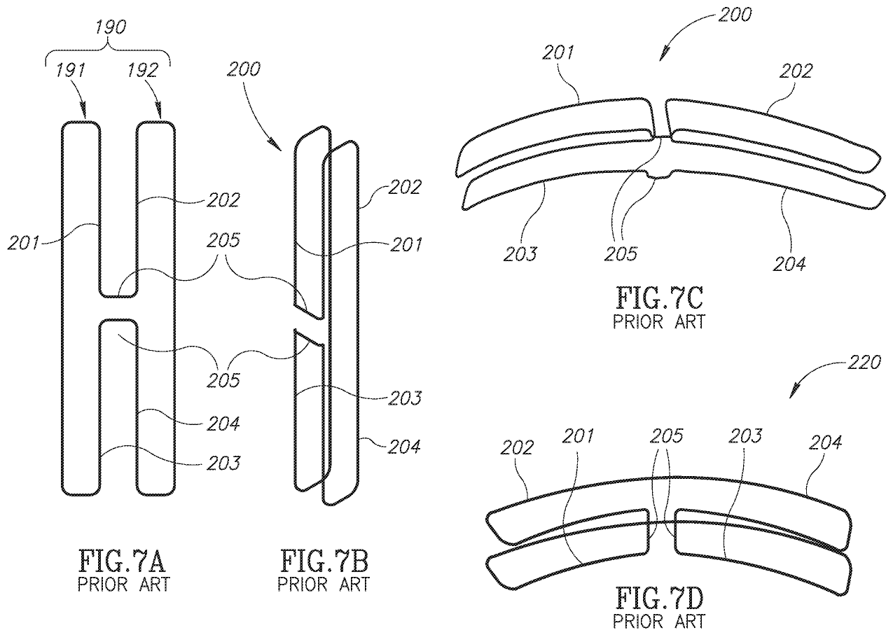

FIGS. 7A-7C schematically illustrate construction of an annular wireframe brace 200, in accordance with an embodiment of the disclosure.

FIG. 7A schematically shows a wireform “blank” 190 after the wireform has optionally been cut from a sheet of a suitable deformable biocompatible material or formed from a wire of such a material, in accordance with an embodiment of the disclosure. Wireform blank 190 optionally comprises two bridge wires 205 that connect wire-loops 191 and 192. Wire-loop 191 comprises top and bottom wire gripping wings 201 and 203 respectively. Wire-loop 192 comprises top and bottom wire gripping wings 202 and 204 respectively. Bending wire-loops 191 and 192 in regions where the wire-loops meet bridge wires 205 as schematically shown in FIG. 7B produces wireframe annular brace 200 in a delivery configuration. FIG. 7C schematically shows wireframe annular brace 200 in a deployed configuration. It is noted that wireform blank 190 may be folded and conditioned to provide a wireframe annular brace 220 shown in FIG. 7D in which top wire gripping wings are gripping wings 202 and 204 and bottom gripping wings are gripping wings 201 and 203. Wireform annular brace 220 is shown in a deployed configuration in FIG. 7D.

Similarly, to annular brace 100 an annular wire brace in accordance with an embodiment of the disclosure may be formed having gripping teeth and stabilizer teeth. And whereas wire-loops 191 and 192 are shown as simple wire loops formed from straight wire sections, wire-loops in accordance with an embodiment of the disclosure may be formed from wavy wire sections or may comprise a wire mesh.

FIG. 8A schematically shows a cardiac enhancer 800 optionally shaped as an integral part for example by sintering, molding or printing from a suitable biocompatible material or by stamping, milling, or laser cutting from a sheet of biocompatible material, in accordance with an embodiment of the disclosure. Optionally, the biocompatible material is a shape memory material which may be a shape memory alloy, such as nitinol, or a shape memory polymer. Enhancer 800 comprises a cardiac brace 100* and a cardiac valve leaflet scaffolding (CAVALS) comprising scaffolding struts 801, 802, and 803 in accordance with an embodiment of the disclosure. Brace 100* is optionally similar to and functions similarly to brace 100 schematically shown in FIGS. 4A-4I. FIG. 8B schematically shows enhancer 800 after folding to the delivery configuration, similarly, as described with respect to brace 100 shown in FIG. 4B. FIG. 8C schematical shows enhancer 800 with brace 100* in a deployed configuration prior to unfolding of scaffolding struts 801, 802 and 803 to their respective deployed configuration. FIG. 8D schematical shows enhancer 800 in a fully deployed configuration with brace 100* and scaffolding struts 801, 802 and 803 in their respective deployed configurations.

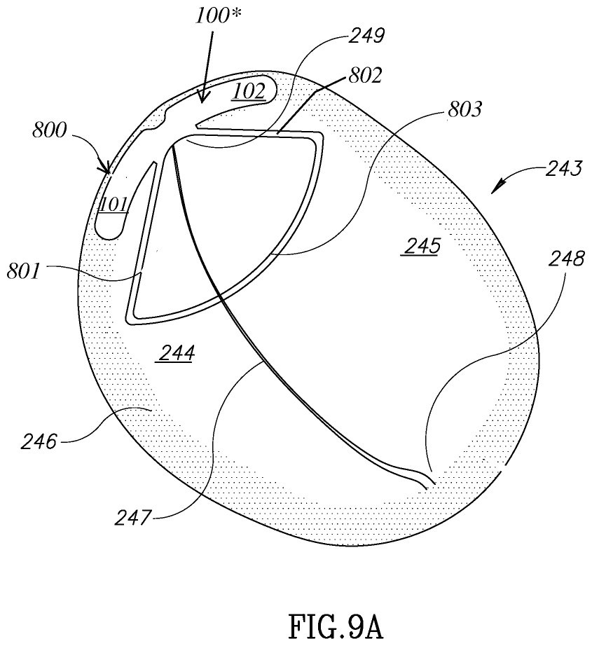

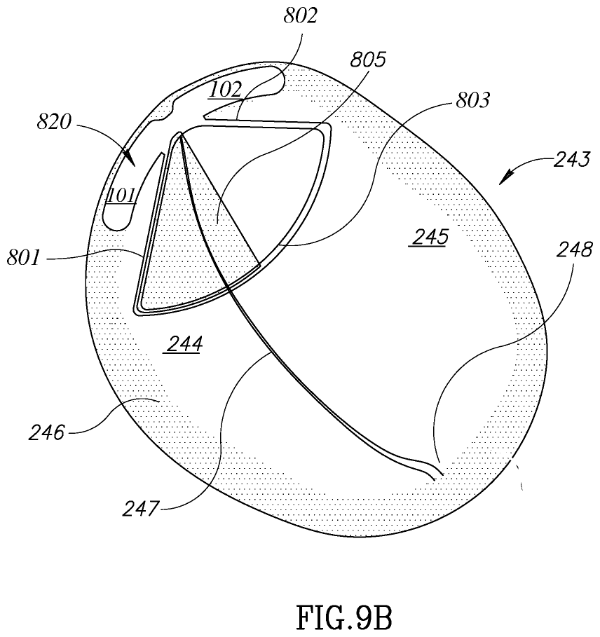

FIG. 9A schematically shows a fully deployed enhancer 800 from an atrial side of a mitral valve 243 with brace 100* anchored to annulus 246 at a commissure of 249 of the mitral valve and scaffolding struts deployed to prevent regurgitation of leaflets 245 and 244 of the mitral valve, in accordance with an embodiment of the disclosure.

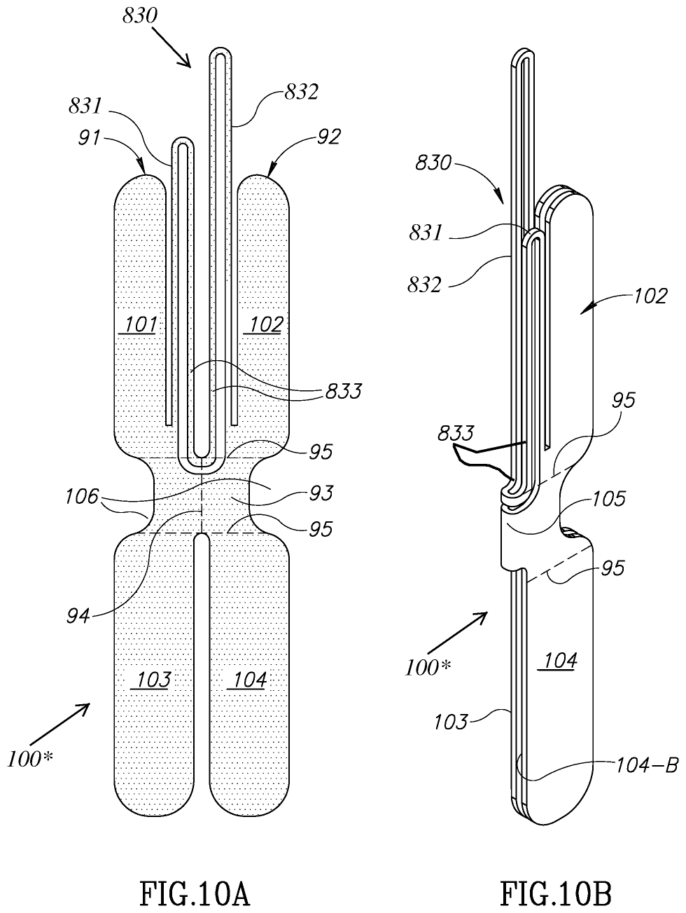

FIG. 9B schematically shows a fully deployed enhancer 820 from an atrial side of mitral valve 243, in accordance with an embodiment of the disclosure. Enhancer 820 is similar to enhancer 800 but comprises a tissue or artificial fabric membrane, hereinafter also referred to as an occluder 805, attached to scaffolding strut 801 and a portion of scaffolding strut 803. Occluder 805 operates to prevent or moderate leakage of blood through a seam 247 between leaflets 245 and 244 at which the leaflet may not coapt properly. In the delivery configuration occluder 805 folds. Optionally accordion-like between scaffolding strut 801 and scaffolding strut 803.

Whereas enhancers 800 and 820 comprise symmetric configurations of three scaffolding struts, embodiments of the disclosure are not limited to symmetric strut configurations or only three struts. For example, FIGS. 10A schematically shows an enhancer 830 in an unfolded state and FIG. 10B schematically shows the enhancer shown in FIG. 10A after folding in a delivery configuration. Enhancer 800 has an asymmetric configuration of scaffolding struts comprising a relative long scaffolding strut 832 bridged to a relatively short scaffolding strut 831 by a scaffolding strut 833, in accordance with an embodiment of the disclosure. The asymmetric strut configuration of enhancer 830 results in the asymmetric configuration of enhancer 830 lying over regions of leaflets 244 and 245 of mitral valve 243 shown in FIG. 9B that are different from the regions overlaid by the symmetric strut configuration of enhancer 800 or 820 and enables enhancer 830 to affect operation of the leaflets and valve 243 differently than enhancer 800 or 820.

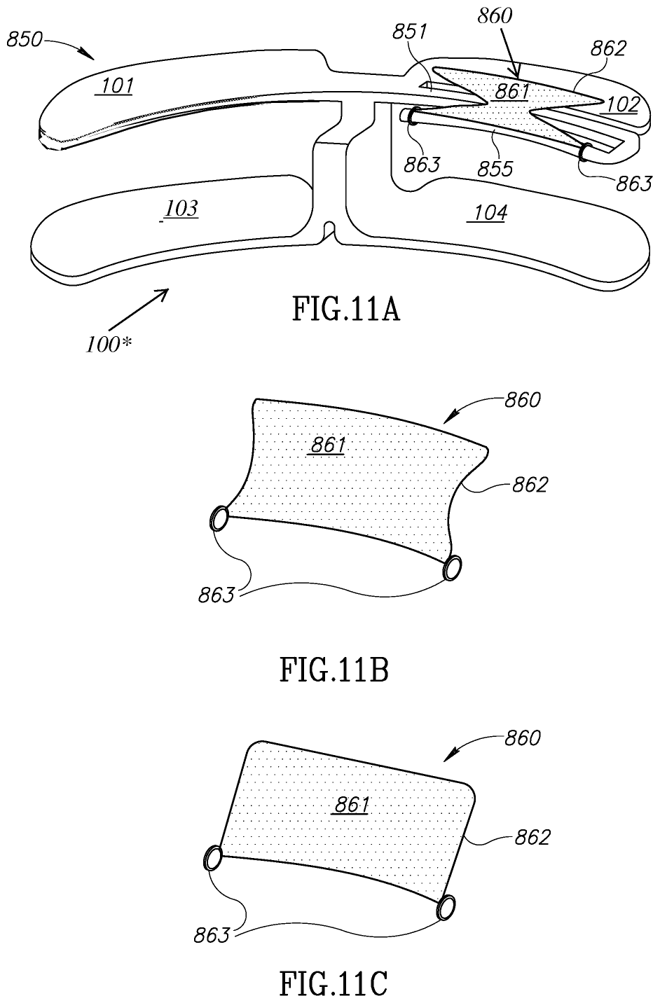

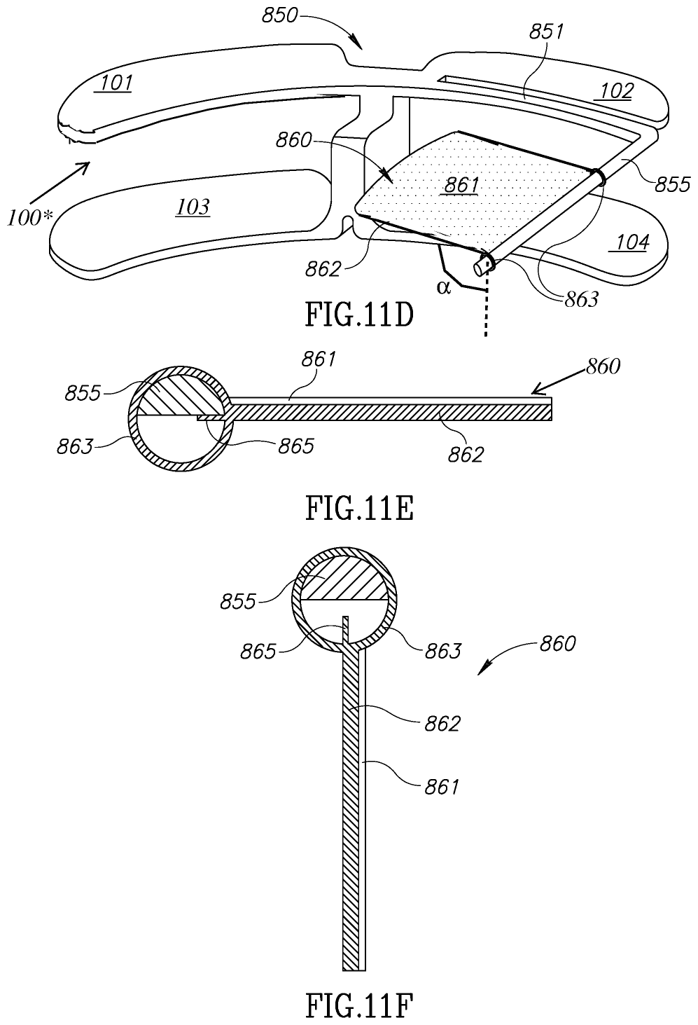

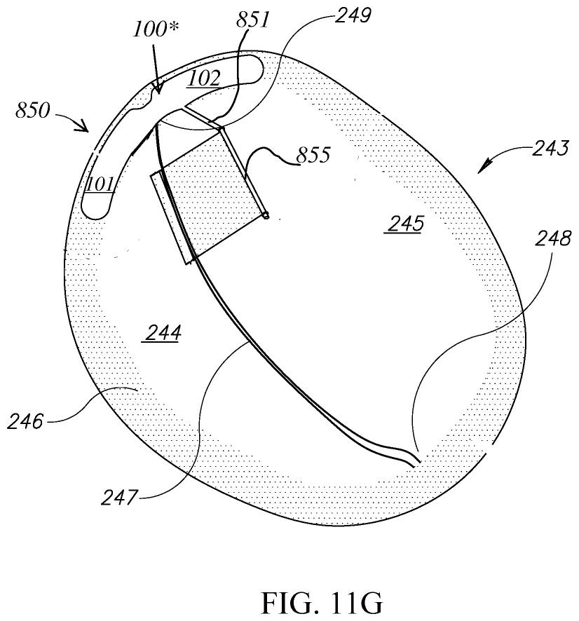

FIG. 11A schematically shows an enhancer 850 comprising a two strut scaffolding comprising a strut 851 and 855. In the figure brace 100* of the enhancer is shown in a deployed configuration having the strut configuration in a delivery configuration before unfolding to a deployed configuration. Optionally, an artificial patch leaflet 860 is rotatably attached to strut 855 by collars 863. Patch leaflet 860 optionally comprises a patch wireframe 862 to which a tissue or artificial fabric membrane, an occluder 861, is attached and is shown in a delivery configuration in which the patch leaflet is folded accordion-like, optionally along a top side of gripping wing 102. In an embodiment patch wireframe is formed from a shape memory material that unfolds to an optionally substantially rectangular deployed shape and unfurl occluder 861 when enhancer 850 is deployed to a cardiac valve. FIGS. 11B and 11C schematically show patch leaflet 860 in a partial and completely unfolded state.

FIG. 11D schematically shows enhancer 850 in a deployed state with both brace 100*, scaffolding struts 851 and 855, and patch leaflet 860 unfolded to their respective deployed states. In an embodiment collars 863 are configured to attach patch leaflet 860 to scaffolding strut 855 and allow the patch leaflet to rotate freely about strut 855 through a limited angle α optionally equal to about 90°. The limitation of angle of rotation of patch leaflet 860 to an angle α prevents the leaflet from rotating “upwards” away from the plane of top gripping wing 102 and facilitates the patch leaflet in preventing regurgitation a cardiac valve to which enhancer 850 is deployed.

FIGS. 11E and 11F schematically show a cross section of a configuration of collar 863 and a region of strut 855 to which the collar attaches patch leaflet 860 that operates to limit rotation of the patch leaflet optionally to angle α. FIG. 11E shows the cross section when patch leaflet 860 is substantially parallel to the plane of gripping wing 102 (FIG. 11D). The region of strut 855 to which collar 863 is attached is formed as a circular sector, optionally a 180° sector. Collar 863 surrounds the circular sector of scaffolding strut 855 and allows the collar to rotate about the sector but has a “stop-tooth” 865 that prevents rotation (counterclockwise and “upwards” in FIG. 11E) of the collar and thereby patch leaflet 860 beyond the position shown in FIG. 11E. Stop-tooth 865 does not on the other hand prevent patch leaflet 860 from rotating clockwise and downwards about scaffolding strut 855 from the position shown in FIG. 11E.

FIG. 11G schematically shows a view of enhancer 850 deployed to mitral valve 243 and having patch leaflet overlapping an atrial side portion of leaflet 245 and a portion of seam 247.

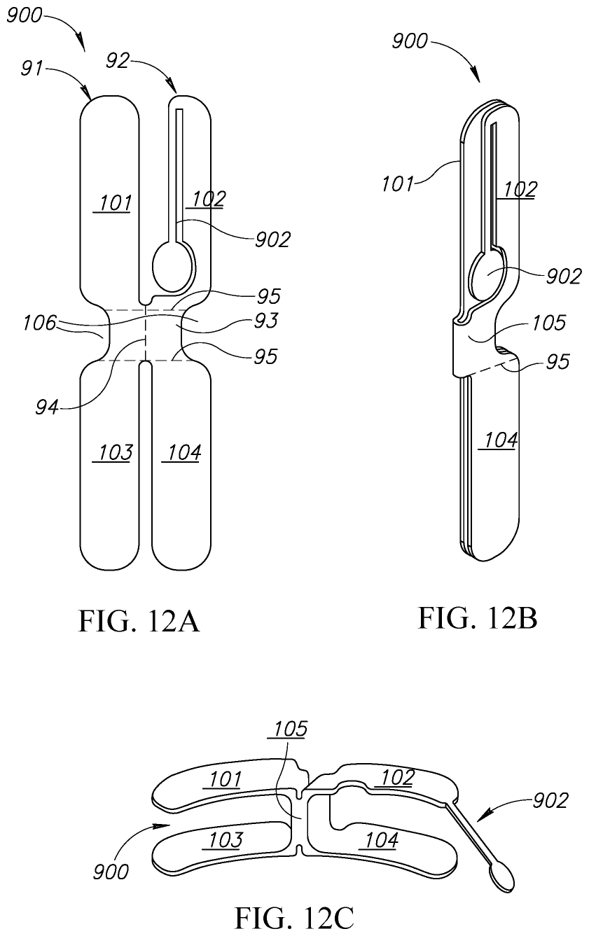

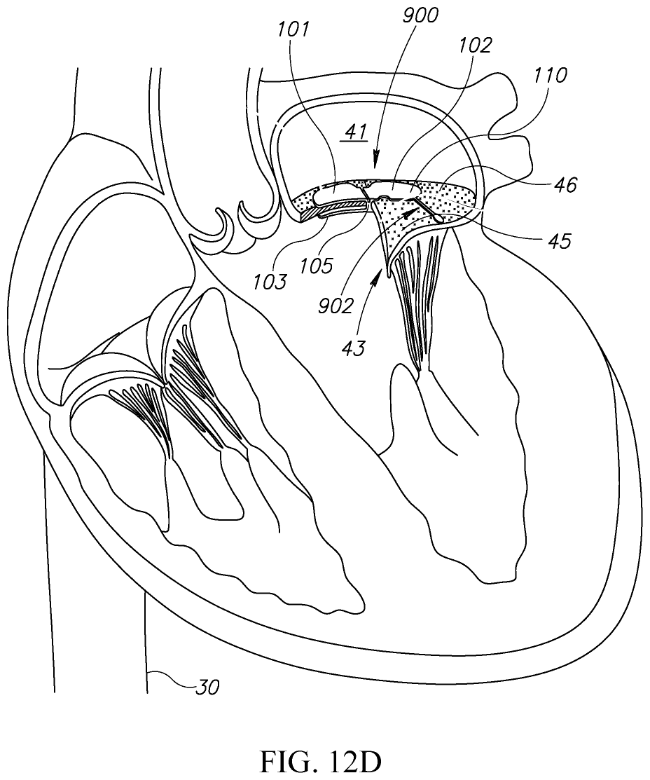

FIGS. 12A and 12D schematically show an enhancer 900 comprising a brace 100* and an integral, extendable leaflet depressor 902, in accordance with an embodiment of the disclosure. FIG. 12A schematically shows enhancer 900 optionally in a form of a planar die-shape before folding. FIG. 12 B shows enhancer 900 after folding in a delivery configuration for delivery by a catheter in accordance with an embodiment of the disclosure. FIG. 12C schematically shows enhancer 900 in a deployed configuration and FIG. 12D schematically shows the enhancer deployed by way of example to a mitral valve 43 in accordance with an embodiment of the disclosure. Depressor 902 is optionally paddle shaped having in the deployed state an angle of extension from gripping wing 102 and an enlarged paddle end to overlay and press on a region of leaflet 45 that is determined to be advantageous for improving operation of the mitral valve. In an embodiment the position and shape of depressor 902 may be determined from finite element analysis of effects of the depressor on motion of the leaflet during systole and diastole.

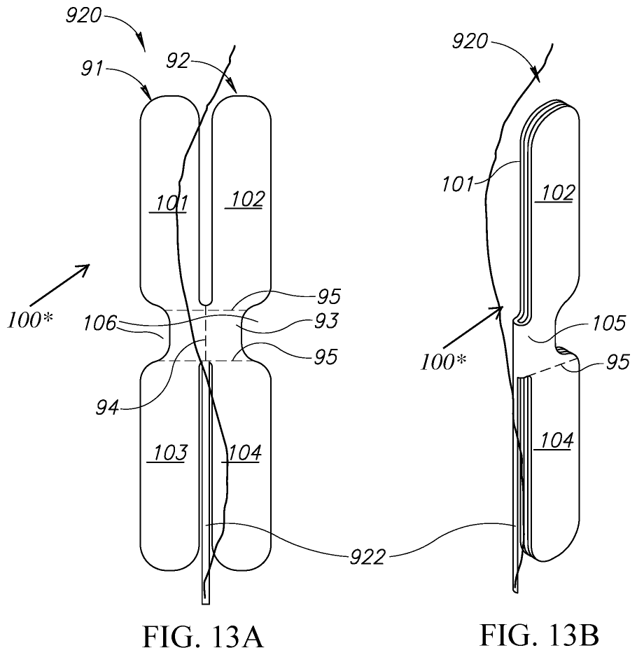

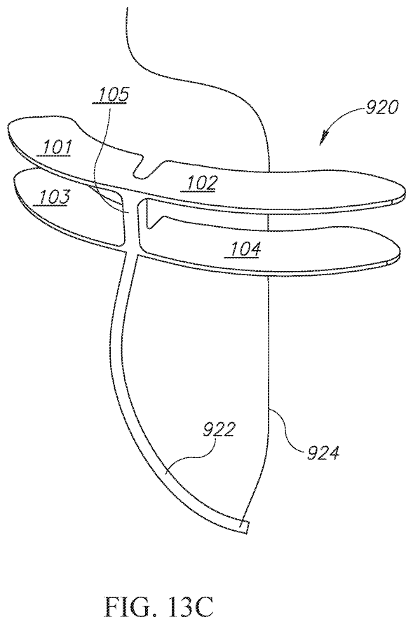

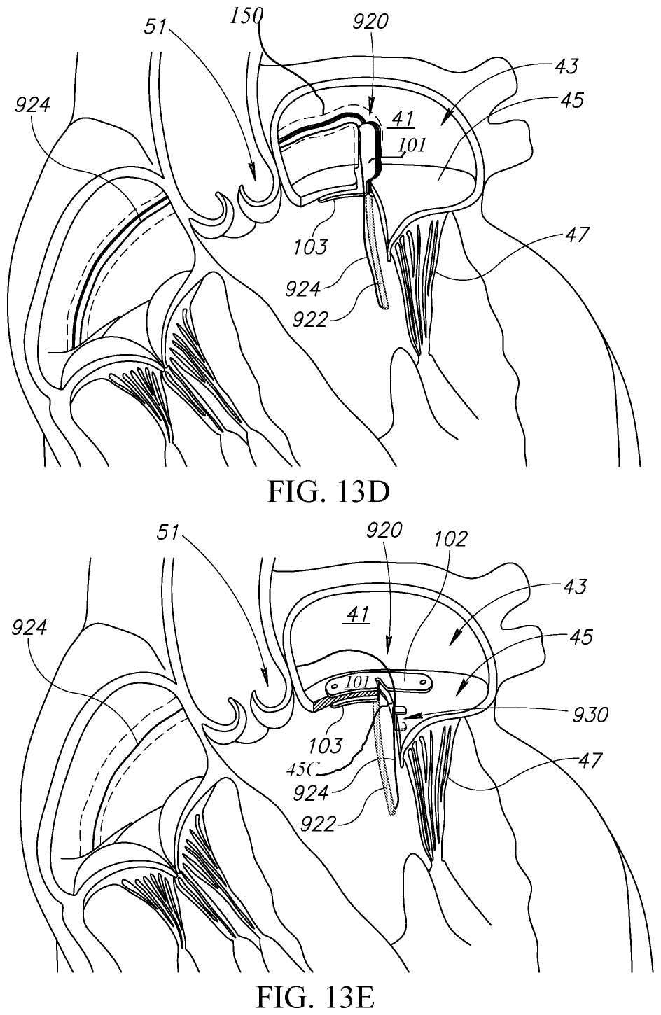

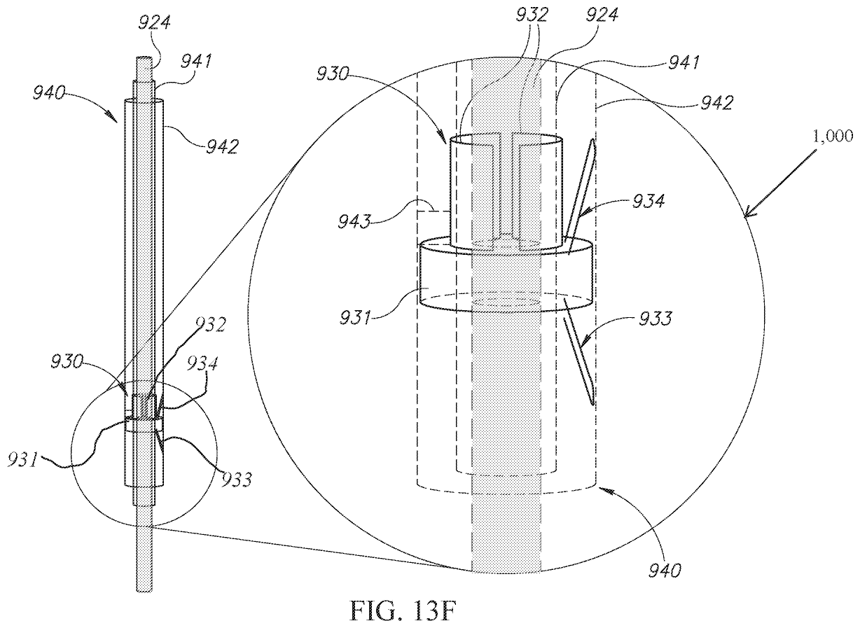

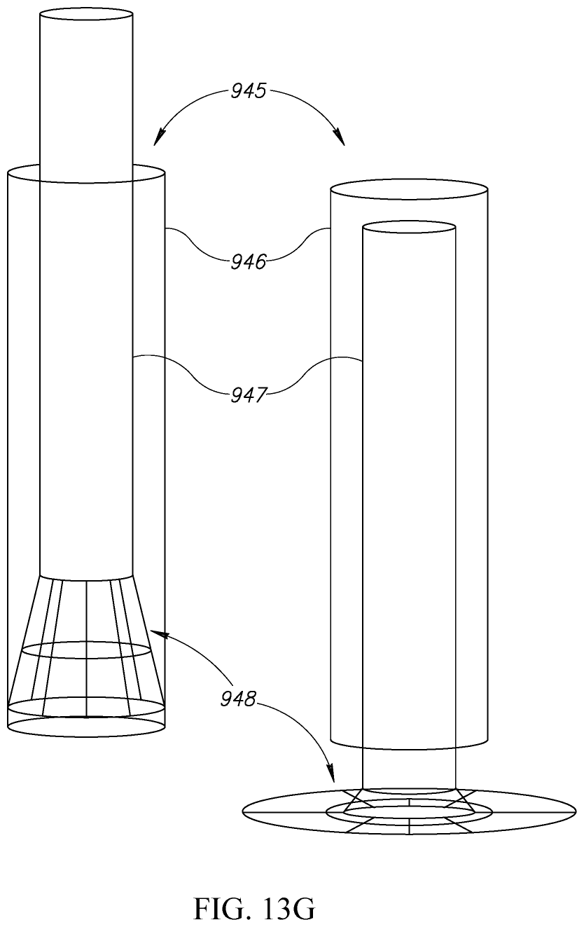

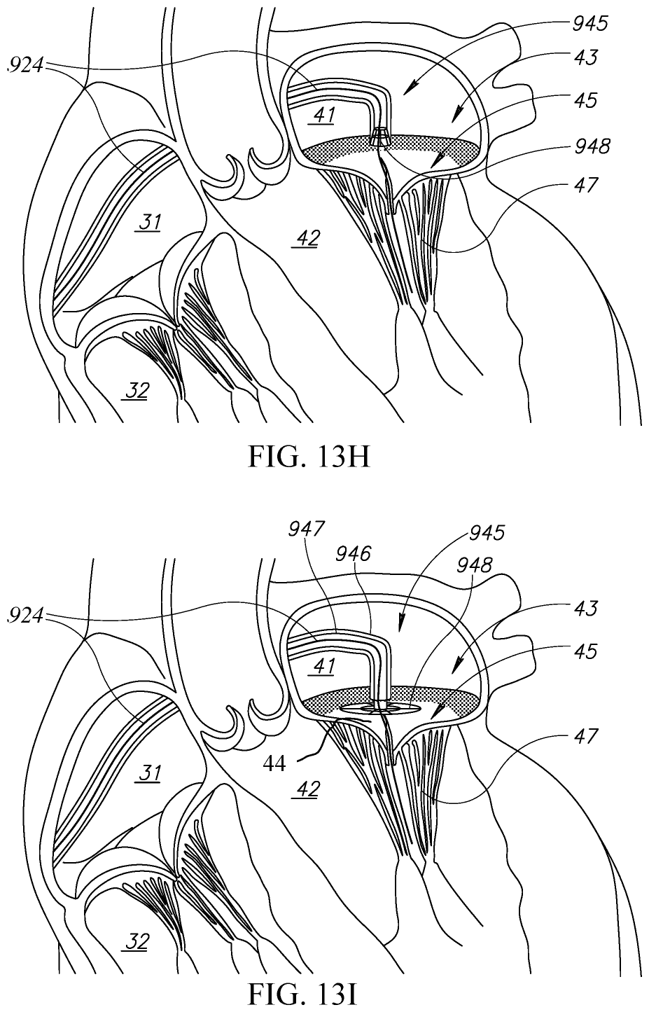

FIGS. 13A-13B schematically shows an enhancer 920 optionally referred to as an emplacer 920 having a delivery configuration for delivery by a delivery catheter (not shown in FIGS. 13A and 13B), and a deployed configuration, which may be used to implant a neochord into the heart to tether a leaflet of a cardiac valve, optionally a mitral valve. FIG. 13A schematically shows schematically shows emplacer 920 optionally in a form of a planar die-shape before folding. FIG. 13 B shows emplacer 920 after folding in a delivery configuration for delivery by a catheter in accordance with an embodiment of the disclosure.

In an embodiment neochord emplacer 920 comprises an annular brace 100* and an extension, also referred to as an anchor tail 922 attached to and extending from bridging region 93 of the brace. A prosthetic neochord 924 for tethering a leaflet of a cardiac valve to which emplacer is deployed is attached to anchor tail 922. In the delivery configuration schematically shown in FIG. 13B the anchor tail optionally extends along the bottom gripping wings 103, 104 of the annular brace and neochord 924 extends along the delivery catheter (not shown) used to deliver the emplacer from a point at which it is attached to the anchor tail to a proximal end of the delivery catheter. FIG. 13 C schematically shows emplacer 920 in a deployed configuration in accordance with an embodiment of the disclosure. When deployed for tethering a leaflet of a mitral valve, the anchor tail extends into the left ventricle.

FIG. 13D schematically shows emplacer 920 being delivered and partially deployed by catheter 150 in a transseptal procedure to a mitral valve 43. FIG. 13E schematically shows delivery catheter 150 partially retracted to fully deploy emplacer 920 to mitral valve 43 and neochord 924 extending between the coaptation edges of mitral valve leaflet 44 (not shown in FIG. 13E) and leaflet 45. A leaflet catcher, such as by way of example a leaflet catcher 930 optionally constructed and deployed as discussed below with reference to FIGS. 13F-13I by sliding along neochord 924, clamps the neochord to a coaptation edge 45C of leaflet 45. After leaflet catcher 930 is deployed to clamp neochord 924 to leaflet 45 excess length of the neochord extending from the leaflet catcher is cut from and removed from the heart.

In an embodiment as schematically shown enlarged in an inset 1000 of FIG. 13F leaflet catcher 930 optionally comprises a base ring 931 that supports neochord clamping jaws 932. The clamping jaws are configured to clamp to neochord 924 (shown shaded) and leaflet gripping fingers 933 and 934 are configured to clutch and hold a region of a coaption edge 45C of leaflet 45 (FIG. 13E). The leaflet catcher is optionally deployed using a catcher deployment system 940 comprising an inner sliding catheter 941 housed inside a holding catheter 942. Catcher deployment system 940 is configured to be slidingly pushed along neochord 924 with the neochord inside sliding catheter 941 and neochord clamping jaws 932 of the leaflet catcher clamped to the outer surface of sliding catheter 941 and leaflet gripping fingers 933 and 944 pressing against the inner surface of holding catheter 942. When the catcher deployment system is slid along neochord 924 to a location at which the leaflet catcher is to be released to clamp to the neochord and clutch leaflet 45 (FIG. 13E), sliding catheter 941 is retracted to extract the sliding catheter from between neochord clamping jaws 932 and allow the clamping jaws to clamp to the neochord. Holding catheter 942 may then be retracted to release leaflet gripping fingers 933 and 934 to clutch and hold leaflet 45. Optionally, clamping catheter 942 comprises at least one internal holding tooth 943 so that when extracting sliding catheter 941 from between neochord clamping jaws 932, the at least one holding tooth operates to prevent leaflet catcher 930 from being extracted with the sliding catheter. Once leaflet catcher 930 is clamped to neochord 924 holding catheter 942 may be retracted proximally to release leaflet gripping fingers 933 and 934 to clutch leaflet 45. Optionally at least one of leaflet gripping fingers 933 and/or 934 is formed having spines (not shown) that bite into and hold the leaflet when the finger is released to clutch the leaflet.

In an embodiment the catcher deployment system comprises a leaflet catcher positioner system hereinafter also referred to as a leaflet positioner, which is usable to determine where to position leaflet catcher 930 along neochord 924 (FIG. 13E-13F) and thereby a length of the neochord attached by leaflet catcher 930 to the leaflet. A leaflet positioner system 945 in accordance with an embodiment of the disclosure is schematically shown in FIG. 13G.

In an embodiment positioner 945 comprises a skirted catheter 947 having an expandable mesh 948, referred to as a skirt, at a distal end of the skirted catheter and an outer, constraining catheter 946, that constrains the skirt to a collapsed configuration. When skirted catheter 947 is pushed out distally from constraining catheter 946 or the constraining catheter retracted proximally so that the skirt 948 is positioned outside of the constraining catheter, the skirt expands to an expanded state. In the expanded state a portion of the skirt assumes a substantially planar state, which may by way of example be a disklike planar shape or a sector of a planar disklike shape

To use positioner 945 to position the leaflet catcher, the positioner is pushed into left atrium 41, as schematically shown in FIG. 13H, using neochord 924 as a guide. When inside the left atrium, as schematically shown in FIG. 13I, constraining catheter 946 is retracted proximally to release skirt 948 to its expanded state. In the expanded state skirted catheter 947 is pushed to bring expanded skirt 948 into contact with the atrial, retrograde sides of mitral valve leaflets 44 and 45 to maintain the leaflets at a desired location during systole. Once in position in contact with the leaflets leaflet catcher deployment system 940 (FIG. 13F) is pushed through skirted catheter 947 to position leaflet catcher 930 relative to expanded skirt 948 to clamp the leaflet catcher at a desired location of the leaflet.

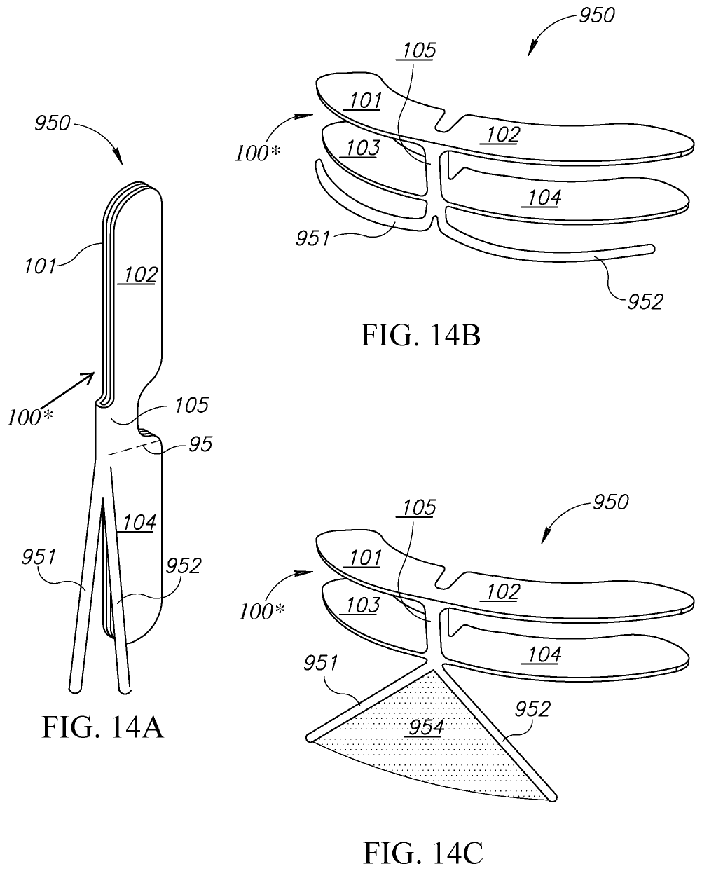

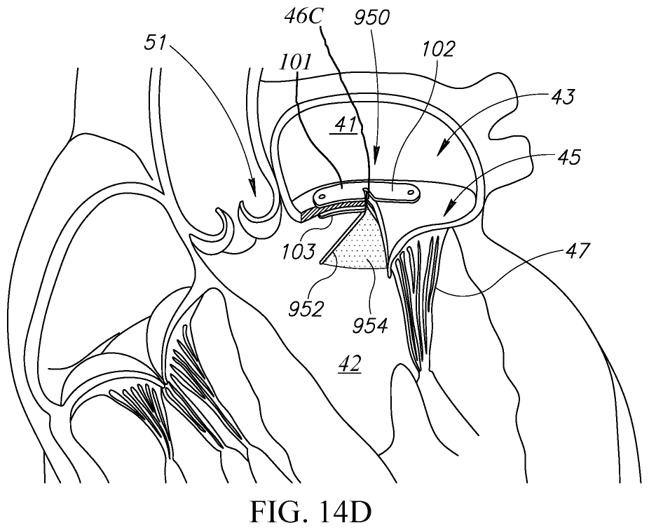

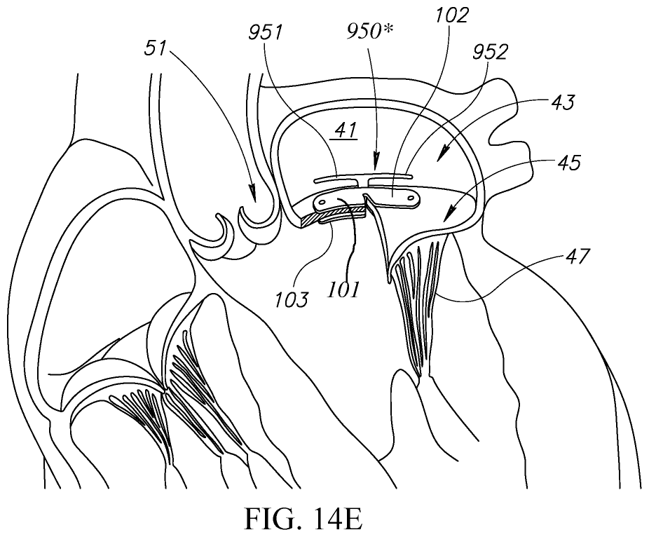

FIGS. 14A-14C schematically show an enhancer 950 optionally referred to as a cardiac modulator (CARMO) that may be deployed in the heart to modulate structure and motion of the heart to advantageously affect functioning of a cardiac valve, in accordance with an embodiment of the disclosure. CARMO 950 optionally comprises an annular brace 100* similar to annular brace 100 and at least one scaffolding strut referred to as a modulator strut, which as shown in FIGS. 14A-14C may comprise two modulator struts 951 and 952 optionally extending from bridge region 105 of brace 100* along bottom gripper wings 103 and 104, in accordance with an embodiment of the disclosure.

FIG. 14A schematically shows CARMO 950 in a delivery configuration. FIG. 14B schematically shows CARMO 950 in a deployed configuration. In an embodiment, as schematically shown in FIG. 14C a foldable web 954 may be attached between modulator struts 951 and 952. In the delivery configuration of CARMO 950 web 954 is folded between struts 951 and 952 and when the modulator struts splay open to their deployed configuration as shown for example in FIG. 14C the web unfurls. FIG. 14D schematically shows CARMO 950 deployed in a mitral valve 43 so that modulator struts 951 and 952 extend into left ventricle 42 to press against apply moderate force to the ventricle wall near commissure 46 of the valve to affect operation of the valve. Web 954 operates advantageously to distributes force applied by modulator struts to the wall.

Whereas CARMO 950 is shown in FIGS. 14A-14D as having modulator struts 951 and 952 lying along gripper wings 103 and 104 in the delivery configuration and deployed to exert force on the ventricle wall, a CARMO 950* in accordance with an embodiment may have modulator struts 951 and 952 lying along upper gripper wings 103 and 104 in the delivery configuration. CARMO 950* may be deployed to mitral valve 41, as schematically shown in FIG. 14E with the modulator struts extending into left atrium 41 and pressing against the wall of the atrium to affect operation of the mitral valve.

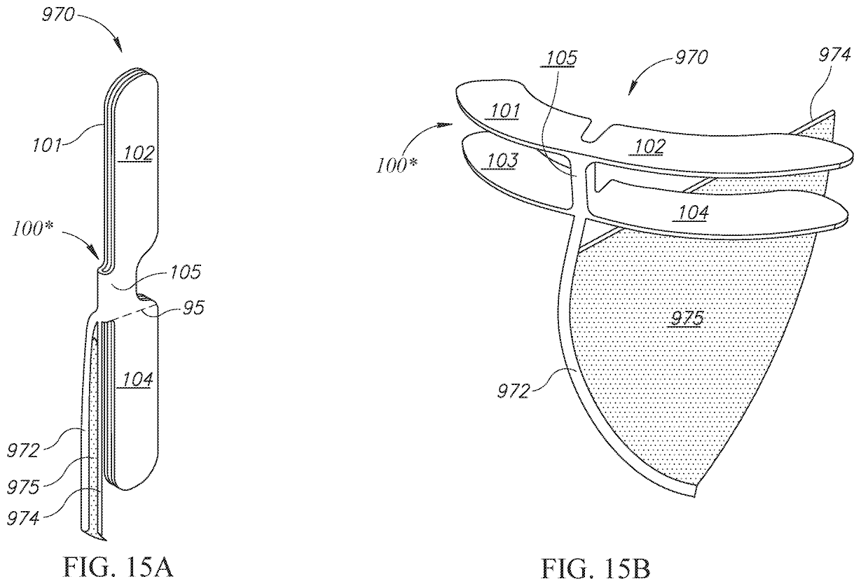

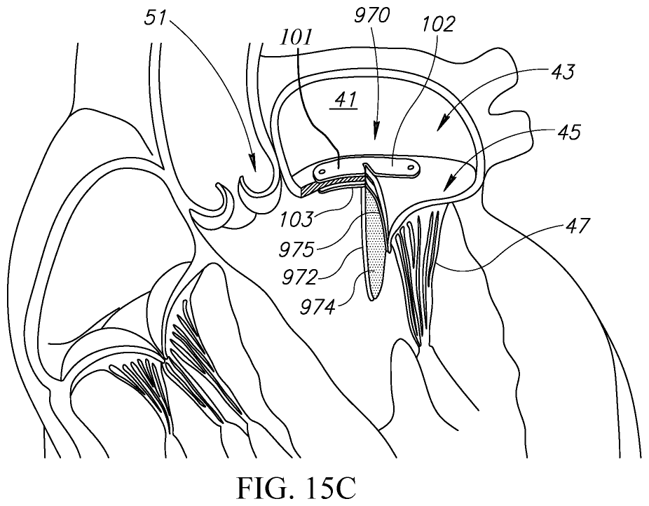

FIGS. 15A-15B schematically show a shim seater 970 in accordance with an embodiment of the disclosure comprising an annular brace similar to annular brace 100 and a scaffolding comprising two struts 972 and 974 connected to a layer 975 of a biocompatible material. In accordance with an embodiment of the disclosure layer 975 has thickness sufficient to fill a lacuna between coapting leaflets of a valve to which the shim seater is deployed and through which lacuna blood regurgitates during systole. FIG. 15A schematically shows shim seater 970 in the delivery configuration and FIG. 15B schematically shows the shim seater in the deployed configuration.

In a delivery configuration, schematically shown in FIG. 15A struts 972 and 974 are folded along bottom gripping wings 103 and 104 of brace 100 with layer 975 folded between them. For example, layer 975 may assume a folded accordion-like configuration in the delivery configuration to fold between struts 972 and 974. In a deployed configuration schematically shown in FIG. 15B struts 972 and 974 may extend to assume a wishbone-like configuration with layer 975 held between them as schematically in FIGS. 15B. When deployed to a cardiac valve, layer 975 seats between two leaflets so that in diastole the layer is squeezed between the leaflets to aid the leaflets to seal against retrograde blood flow. FIG. 15C schematically shows shim seater 975 deployed to a mitral valve 43 and layer 975 and positioned at a location between leaflet 45 and leaflet 44 (FIG. 2A) of the valve.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

Descriptions of embodiments of the disclosure in the present application are provided by way of example and are not intended to limit the scope of the disclosure. The described embodiments comprise different features, not all of which are required in all embodiments of the disclosure. Some embodiments utilize only some of the features or possible combinations of the features. Variations of embodiments of the disclosure that are described, and embodiments of the disclosure comprising different combinations of features noted in the described embodiments, will occur to persons of the art. The scope of the disclosure is limited only by the claims.

1. Apparatus for affecting operation of a cardiac valve of a heart, the apparatus comprising:
an annular cardiac brace for mounting to an annulus of a cardiac valve having cardiac valve leaflets, the brace having:
first and second bottom gripping wings for gripping an annulus of a cardiac valve;
first and second top gripping wings for gripping the annulus;
a support bridge that connects the top wings to the bottom wings; and
a scaffolding connected to the brace and comprising at least one extendable scaffolding strut;
wherein the apparatus is deformable from a delivery configuration to a deployed configuration and in the delivery configuration the top wings are oriented substantially back to back along an axis, and the bottom wings are oriented substantially back to back along the same axis and the at least one extendable scaffolding strut lies along the top or bottom wings, and in the deployed configuration the first top and bottom gripping wings face each other to grip the annulus between them and the second top and bottom gripping wings face each other to grip the annulus between them and the at least one extendable scaffolding arm extends away from the wings into a chamber of the heart on a retrograde side of the valve or an antegrade side of the valve.

2. Apparatus according to claim 1 wherein in the delivery configuration the at least one scaffolding strut is folded towards the top gripping wings.

3. Apparatus according to claim 2 wherein in the deployed configuration the at least one scaffolding strut unfolds to extend from the brace into the retrograde side of the cardiac valve.

4. Apparatus according to claim 3 wherein in the deployed configuration the at least one scaffolding strut contacts a retrograde side of a leaflet of the valve during systole.

5. Apparatus according to claim 4 wherein the at least one scaffolding strut comprises a single strut.

6. Apparatus according to claim 5 wherein the single strut is paddle shape having an enlarged paddle end that presses on a region of a leaflet of the valve during systole.

7. Apparatus according to claim 2 wherein the at least one scaffolding strut comprises a plurality of connected struts.

8. Apparatus according to claim 7 wherein the plurality of struts are connected to delineate and surround a closed area.

9. Apparatus according to claim 8 wherein the enclosed area overlays a seam of the valve between leaflets of the valve along which the leaflets in a healthy valve close during systole.

10. Apparatus according to claim 2 wherein in the deployed configuration the at least one scaffolding strut unfolds to extend from the brace to press on a wall of an atrial chamber of the heart.

11. Apparatus according to claim 1 wherein in the delivery configuration the at least one scaffolding strut is folded towards the bottom gripping wings.

12. Apparatus according to claim 11 wherein in the deployed configuration the at least one scaffolding strut unfolds to extend from the brace into the antegrade side of the cardiac valve.

13. Apparatus according to claim 12 wherein the at least one scaffolding strut extends from the brace to press on a wall of a ventricular chamber of the heart

14. Apparatus according to claim 12 and comprising a neochorda connected to the at least one scaffolding strut.

15. Apparatus according to claim 11 wherein the at least one strut comprises at least two struts.

16. Apparatus according to claim 15 wherein the at least two struts support a biocompatible material.

17. Apparatus according to claim 16 wherein the at least two struts and the layer of material lie between two leaflets of the valve and during systole the layer of material is sandwiched between the leaflets.

18. Apparatus according to claim 17 wherein the layer of biocompatible material has thickness sufficient to fill a lacuna between two leaflets of the valve and through which blood regurgitates during systole.

19. Apparatus according to claim 1 wherein the brace is integrally formed as a single piece from a same material.

20. Apparatus according to claim 1 wherein the brace and scaffolding are integrally formed as a single piece from a same material.

21. Apparatus according to claim 19 wherein the material is a shape memory material.
